(19) United States
(12) Patent Application Publication
(10) Pub. No.: US 2015/0080468 A1
(43) Pub. Date: Mar. 19, 2015

(54) COMPOSITION AND METHOD FOR PROCESSING AND DELIVERING BIOAVAILABLE METHIONINE ANALOGS, DERIVATIVES, AND PRECURSORS, THEREOF

(71) Applicant: QUALITY LIQUID FEEDS, Dodgeville, WI (US)

(72) Inventors: Steve Freeman, Dodgeville, WI (US); Lisa Davis, Dodgeville, WI (US); Howard Blalock, Dodgeville, WI (US)

(73) Assignee: QUALITY LIQUID FEEDS, Dodgeville, WI (US)

(21) Appl. No.: 14/026,199

(22) Filed: Sep. 13, 2013

(51) Int. Cl. A61K 31/198; A23K 1/18; A23K 1/16

(52) U.S. Cl. CPC A61K 31/198; A23K 1/1634; A23K 1/1813
USPC 514/562

(57) ABSTRACT

Methionine products/additives are added to dairy cattle feeds to promote milk volume production and to enhance milk components. Unfortunately the methionine products/additives solidify/crystallize at about 54° F. (12° C.), making the products/additives difficult to dispense in a liquid storage and pumping system in cold conditions, thus requiring a time-consuming, expensive process using specialty equipment to de-crystallize the product. In one aspect this invention is an admixture that keeps the methionine products/additives temperature at a level that does not crystallize and thus, eliminates the expensive and time-consuming drawbacks to using methionine products in colder climates.

### FIELD OF THE INVENTION

The present invention relates to compositions and methods for processing and delivering bioavailable liquid methionine and related analogs of methionine. More specifically the present invention relates to methods and compositions which inhibit the crystallization of methionine analogs which occurs at temperatures in the range of 54° F. (12° C.) and below. By virtue of this invention delivery of bioavailable liquid methionine and related analogs of methionine is obtained at substantially lower storage temperatures and delivery costs and with substantially greater convenience to both processors and consumers of the liquid methionine and related analogs of methionine.

### BACKGROUND

Protein is one of the major nutrients in the diets of lactating cows. The cows however do not actually require proteins but instead they require the specific amino acids, which are the building blocks used to make up their own protein.

It is known that methionine is a limiting amino acid, and in particular for milk production, it is believed that a well-balanced level of methionine will result in effective levels of milk production. It is also believed that an increase in methionine levels can result in increased milk production.

Methionine is added to e.g., dairy cattle feeds, to promote milk volume production and to enhance the quality of milk components. Methionine has been added to dairy cattle diets directly as a dry feed article either in a meal or pellet form or as a liquid. Liquid methionine and related analogs of methionine, which are liquid at room temperature and atmospheric pressure, have a crystallization temperature of about 54° F. (12° C.). At that temperature and just below, its handling characteristics and viscosity significantly change making the additive impossible to dispense in a liquid storage and pumping system because it has solidified. It goes through a process known as superfusion, where the liquid methionine changes from a liquid to a solid. A heated, environmentally enclosed storage and pumping system is required to keep the liquid methionine and related analogs fluid and pumpable at a temperature greater than 54° F. (12° C.) i.e., its crystallization temperature or above.

Ambient cold storage temperature conditions often exist in feed mills and at dairies which limit the potential use of these products and/or necessitate investment in drying these products on special dry carriers or providing energy to heat and special storage equipment to provide flowability for practical applications as mentioned above. The invention provides for low cost flowability of high concentrations of liquid bioavailable methionine products stored under cold conditions in conventional storage and pumping systems used for liquid supplements typically employed in dairy and livestock operations.

For example, a commercial methionine sold under the designation MetaSmart™ by Adisseo, a Bluestar company. The MetaSmart™ User's Guide indicates “MetaSmart™ can crystallize below 54° F. (12° C.) to ensure proper fluidity.” The User's Guide includes extensive description of heated tanks and heating assemblies needed to prevent MetaSmart™ methionine from crystallizing.

### BRIEF SUMMARY OF THE INVENTION

Briefly, in one aspect, this invention provides for low cost flowability of high concentrations of liquid bioavailable methionine products and related analogs of methionine under cold storage conditions through addition of said compounds to liquid mill/feed ingredients formulations. Liquid mill/feed ingredient formulations with added high concentrations of liquid bioavailable methionine and related analogs of methionine dramatically lower the crystallization point to allow storage and subsequent flowability under cold temperature conditions found under normal feed mill storage conditions.

This invention also replaces the need to purchase more expensive liquid bioavailable methionine products that have been dried onto special carriers or to the purchase of energy to heat such products and the special equipment necessary to provide flowability for practical applications. Liquid bioavailable methionine dried on special carriers or the use of heating and special equipment to maintain flowability of said products results in higher costs of handling and maintaining product usefulness than said invention.

For purposes of this invention the following definitions apply:

“Methionine” and “methionine products” are broadly interpreted to mean methionine itself, derivatives, precursors (e.g., metabolic precursors), analogues and any other molecule or amino acid which exhibits nutritional characteristics, e.g., for animal feed, having a methionine structure or backbone and which exhibit storage processing or handling characteristics e.g., crystallization temperatures, that this invention reduces or eliminates. Specifically included in this definition, without limitation, are esters, amides, a hydroxy analogues, methionine precursors and structurally related molecules. The various derivatives of methionine discussed in U.S. Pat. No. 6,221,909 to Robert et al. are specifically incorporated by reference herein as being within the meaning of this term.

The term “liquid mill/feed ingredient formulations” or its substantial equivalent “liquid feed product” for purposes of this invention means, without limitation, condensed fermentation solubles, corn steep liquor, or distillers solubles or molasses. Liquid feed ingredients include both products and co-products of fermentation processes, distiller's products, brewer's products, grain processing, milk products, paper or wood manufacturing, or molasses products. Operant liquid mill/feed ingredient formulations must be liquid within the broad range of ambient temperatures to which such formulation are exposed.

For the purposes of this application, “crystallization point suppression”, Δt_{c}, is the number of degrees (either ° F. or ° C.) that the crystallization temperature of methionine or methionine products is reduced (i.e., Δ) by application of this invention. “Crystallization temperature”, for purposes of this invention, is the temperature at which methionine or methionine products crystallize out of solution at ambient pressure.

### BRIEF DESCRIPTION OF THE DRAWINGS

A better understanding of the present invention as well as other objects and advantages thereof will become apparent upon consideration of the detailed description, especially when taken with the accompanying drawings wherein like numerals designate like parts throughout, and wherein:

FIG. 1 is a view of the bench samples of the test formulas 24-hr after manufacturing.

FIG. 2 is a view of the refrigerator samples of the test formulas 24-hr. after manufacturing.

FIG. 3. Time, Concentration, and Mixing or Static Affects on Bench Sample pH.

FIG. 4. Time, Concentration, and Mixing or Static Affects on Refrigerator Sample pH.

FIG. 5. Viscosity as Affected by Temperature, HBMi Concentration, and Mixing Action during the 56 Day Study.

FIG. 6. HMBi in a state of superfusion after being in sub-zero freezer for eight days and container being shaken to cause it to solidify.

FIG. 7. Effect of Temperature on 8.8% HMBi liquid formulation viscosity over time.

FIG. 8. Effect of Temperature on 17.6% HMBi liquid formulation viscosity over time.

### DETAILED DESCRIPTION OF THE PREFERRED EMBODIMENT

In one aspect, this invention comprises an intimate composition or admixture of a high concentration of liquid methionine product with a liquid mill/feed ingredient formulation. The admixture is intimate in the sense that methionine crystallization (superfusion) is suppressed at least 10° F. (5.6° C.) (i.e., a crystallization temperature change, a Δt_{c}, of 10° F. (or 5.6° C.)), preferably at least 18° F. (10° C.) (i.e., at Δt_{c}, of 18° F. (or 10° C.)), and most preferably a Δt_{c} of at least 24° F. (13.3° C.) (i.e., 24° F. (or 13.3° C.)). Other than the suppression of liquid methionine product crystallization temperature as required herein, the precise nature of the interaction between the liquid bioavailable methionine product and the liquid mill feed product is not critical.

The present invention admixture eliminates the need for specialized and expensive equipment to handle the liquid methionine product alone and allows for the admixture to contain high concentrations of the liquid methionine product that will remain flowable and pumpable at temperatures approaching 30° F. (−1° C.). Typical liquid mill/feed supplement product equipment can be used to store and manage the admixture. Ambient temperatures below 30° F. (−1° C.) will require minimal heating to maintain admixture temperature at and just above 30° F. (−1° C.) to allow pumping of admixture.

A preferred liquid methionine product in the practice of this invention is a product referred to as MetaSmart™ which is commercially available from the Adisseo company. MetaSmart™ is characterized by the Adisseo Company as follows:

### DESCRIPTION

MetaSmart™ is a liquid source of bio-available methionine for dairy cows. It is the isopropyl ester of the hydroxylated analogue of methionine, also referred to as HMBi for short.

It is a brown to colorless liquid, exclusively for use in the manufacture of dairy cattle feed products.

### Composition

HMBi (Isopropyl ester of 2-hydroxy-4-methylthiobutanoic acid):

CAS number [57296-04-5]

Chemical formula: CH₃S—(CH₂)₂—CH(OH)—COO—CH—(CH₃)₂

### Specifications

Appearance: Liquid

Color: Brown to colorless

HMBi monomer content: 95% minimum

Water content: 0.5% maximum

Typical Analysis*

pH (in a 1% aqueous solution): 3.6

Relative density: 1.074 kg at 20° C./kg water at 4° C.

Viscosity at 20° C.: 19.2 mPa/s

Solubility: 25.1 g/liter water at 30° C.

Packaging

1000 kg net containers (IBC)

Utilization

Exclusively for animal nutrition, for incorporation into dairy cattle feed.

### Method of Analysis

Internal method, reference number Z 047

Dosage by HPLC/UV using an external calibration.

MetaSmart™ Liquid Technical Information Sheet

### Safety Information

Reference: Material safety data sheet for MetaSmart™, version 1.0.

Nutritional Specifications

MetaSmart™ Liquid provides 370 g/kg of metabolizable methionine and 370 g/kg of rumen available HMB.

Suggested input values for CPM Dairy and NRC 2001 are as follows:

| NRC 2001 | |
|---|---|
| Crude Protein | 95 |
| A | 50 |
| B | 0 |
| C | 50 |
| Methionine, % of CP | 88 |
| RUP digestibility | 100 |

| CPM Dairy | |
|---|---|
| Dry Matter | 95 |
| Crude Protein | 78 |
| Soluble Protein | 100 |
| Methionine, % of RUP | 100 |
| Rates: | |
| Protein A | 10,000 |
| Protein B1 | 8 |
| Protein B2 | 0 |
| Protein B3 | 0 |

This invention will now be illustrated by the following Examples which are intended to be illustrative and not limiting of its scope.

### EXAMPLES

### Example 1

Example 1: A simple liquid mill blend was formulated using molasses products, wet corn-milling co-product, cheese manufacturing co-products, corn based ethanol production co-product (Table E1-1.) and a methionine analogue additive, HMBi; [MetaSmart®, isopropyl ester of 2-hydroxy-4-methylthiobutanoic acid, CAS #57296-04-5; CH₃S—(CH₂)₂—CH(OH)—COO—CH—(CH₃)₂] to investigate the effects of temperature and mixing on the handling, pumping, and stability characteristics of the formulations at two HMBi concentrations in the base mill liquid. Two concentrations of the HMBi were used: 3× or 264 pounds per ton and 4× or 352 pounds per ton formula batch size of mill liquid. Each additive concentration was either placed on the laboratory bench to be kept at room temperature or stored in a typical household refrigerator for cold storage (˜−1° C.) (˜30° F.). Within a temperature environment, each HMBi concentration was either stirred once daily or left un-stirred.

TABLE E1-1
Liquid Mill Product Formulation and
Methionine Analogue Additions

| Ingredient | Inclusion Range % |
|---|---|
| Molasses Product | 32-35 |
| Cheese Co-Products | 19-22 |
| Wet Corn-Milling Co-Product | 13-16 |
| Ethanol Co-Product | 13-16 |
| Methionine Analogue, HMBi | 13.2 or 17.6 |

Therefore, there were eight individual test formulations to monitor over time (Table E1-2).

TABLE E1-2
Liquid Mill Product Test Formulations

| Formulation | Location/Temperature | Action |
|---|---|---|
| F1BM3X | Bench Room Temp | Hand Mixed |
| F2BS3X | Bench Room Temp | Static Not mixed |
| F3RM3X | Refrigerator | Hand Mixed |
| F4RS3X | Refrigerator | Static Not mixed |
| F5BM4X | Bench Room Temp | Hand Mixed |
| F6BS4X | Bench Room Temp | Static Not mixed |
| F7RM4X | Refrigerator | Hand Mixed |
| F8RS4X | Refrigerator | Static Not mixed |

Each test was made individually for eight separate batches of approximately 2,000 grams each. Ingredients were added from the highest inclusion amount to the least. The mixing/observation vessel was a tall PET plastic container with the following dimensions and had a closure to seal the container: diameter 4 1/16 inches by depth 8⅛ inches with approximate capacity of 58 ounces. Mixing was continuous after adding the first ingredient.

Initial mixer speed was approximately 250 rpms. After the last ingredient addition, the mixer was run for an additional 10 minutes at maximum revolutions, 500 rpms. The mixer used was an electric Talboys Laboratory Stirrer, Troemner, LLC, Thorofare, N.J., ¼ hp, 120 v, 250 W, 1.8 amps, 60 Hz, 50-500 rpm variable speed mixer. The mixer blade was a plastic ribbed offset parallel vane paint mixer blade.

All test formula batches were allowed to rest for at least 24 hours before any initial measures or data collection was performed to allow for release of any entrained air due to mixing. Data collected included: Visual appearance to determine degree of positional stability, visual appraisal of microbial activity, smell, viscosity, temperature, pH, and gross flow characteristics. Viscosity was measured using a Brookfield HADVE115 E7106 viscometer and Brookfield HA/HB spindle set with quick release system. The spindle size used was determined by maintaining the torque within 10 to 90% range. Spindle speed was set at 50 rpms with a duration time of one (1) minute before viscometer motor was shut off. Viscosity was read directly from digital display in centipoises (cP) with no conversion because of being able to dial in spindle number with the HADVE115 E7106 unit. The pH was determined using an Orion Star™ Series 3-Star bench top meter, Thermo Electron Corp., with a refillable Ag/AgCl pH electrode 9172BNWP. Temperatures were record using a metal probe type with digital readout.

### Observations and Results

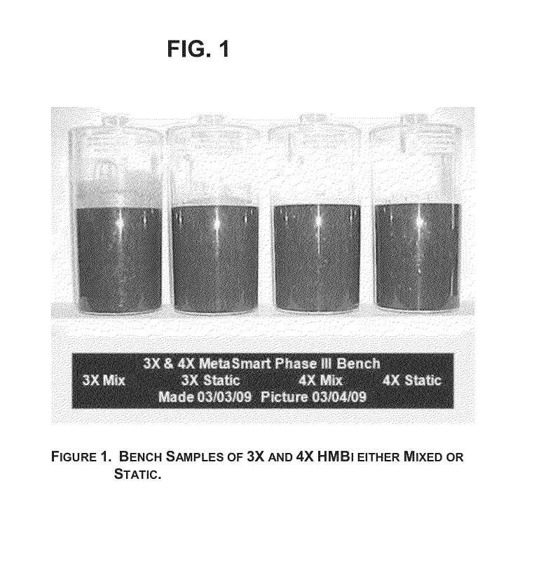

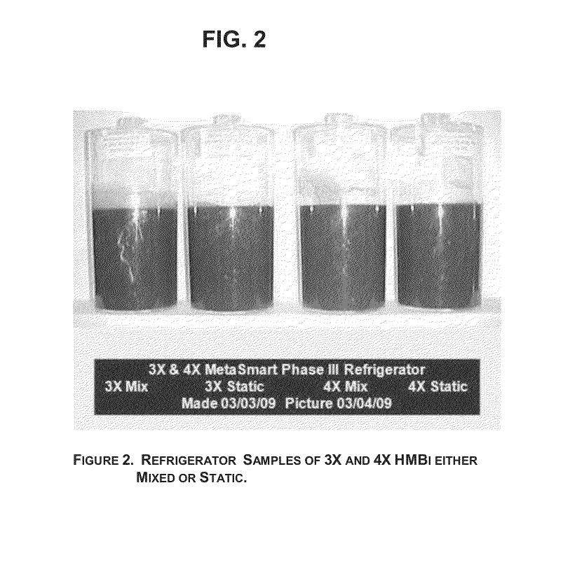

FIG. 1 and FIG. 2 are two views of the test formulas 24-hr after manufacturing. Data was taken over a 56-day period. The samples to be mixed were stirred at least three times every seven days over the observation period. Viscosity, pH, temperatures were taken at staggered intervals. Results are given in Table E1-3a-e. The plain mill liquid product without any HMBi added has a very short inventory shelf life with a high degree of surface microbial activity. Fermentation and surface mold growth occurs within days, making the plain mill product unusable in typical feed mill operations. This was determined in a preliminary study.

During this study, none of the eight samples showed any visible or olfactory signs of fermentation or surface mold growth. HMBi has a very distinctive smell that was very noticeable in all eight samples, with the 4× samples having a stronger odor throughout the 56-day test period. Bottom separation did occur in the static samples after a period of about 14 days with the bench samples for both the 3× and 4× mixes. Bottom separation was greater as indicated by increased depth of a dark less viscous bottom layer for the 3× liquid. It is not readily apparent why the 4× Bench Static sample did not have greater bottom separation compared to the 3× Bench Static sample. None of the mixed samples, regardless of temperature (location), showed signs of separation. Static Refrigerator samples of both the 3× and 4×, showed some very slight bottom separation. These observational results indicate that every other day mixing will keep the product from separating in mild temperatures. Separation, thus mixing, is not an issue when product temperature is about at freezing.

However, inventory kept longer than two weeks should be mixed at least once per week during the colder times of the year and at least three times a week during warm times of the year.

Initially viscosities were similar between the static and mixed samples within a particular HMBi concentration and temperature. However, by two weeks into the study, the mixed samples were thinner regardless of temperature and HMBi concentration. The 4× HMBi mixes were thicker at both temperatures compared to the 3× HMBi formulations (Table E1-3a-e).

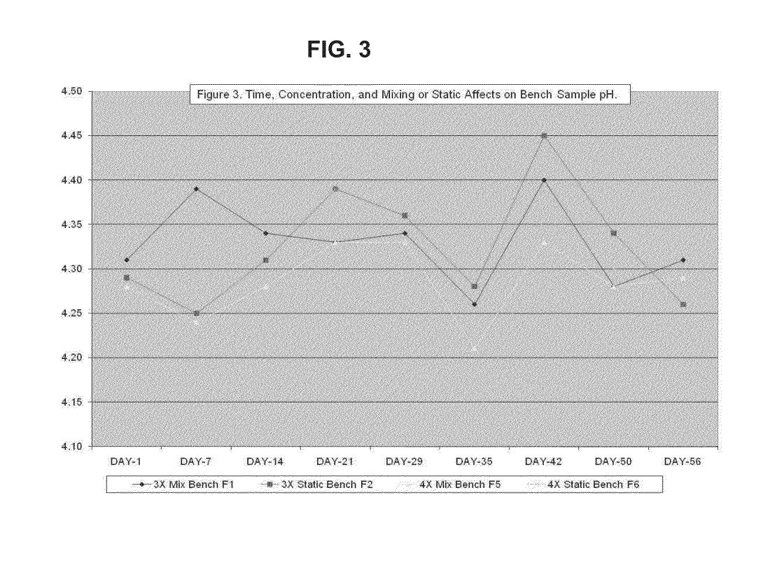

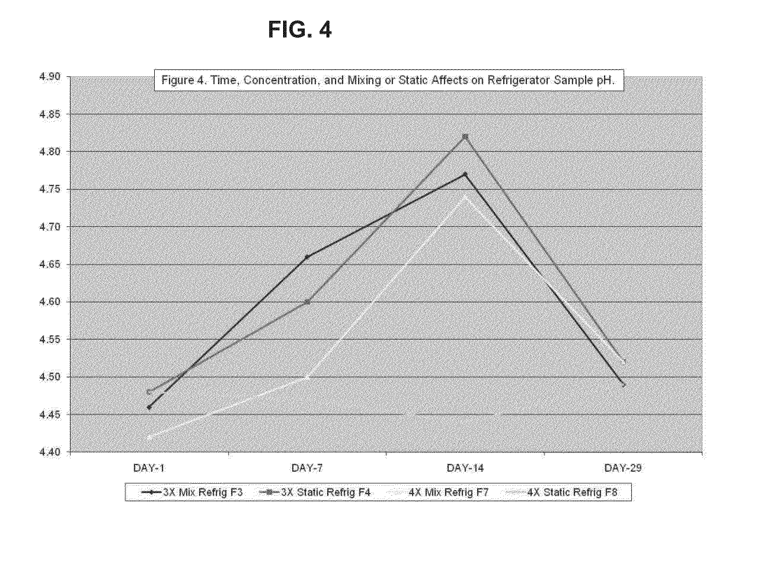

Across HMBi concentrations, the pH did not significantly vary over the 56-day study period. However, it was noted that the refrigerator samples regardless of HMBi concentration, had a more basic pH (4.55 vs 4.31). It was thought that the lower temperature could be the cause of a more basic pH with the refrigerator samples. After 29 days, pH was not measured on the refrigerated samples because of possible interferences due to the lower temperature. The pH values are expressed in FIG. 3 and FIG. 4.

Viscosity and percent torque as affected by time, concentration of HMBi, and mixing or static nature of the samples is given in the following tables: Tables E1-3 a-e. Spindle #2 was used.

TABLE E1-3a
Time, Concentration of HMBi, and Action Effects on
Viscosity and Percent Torque.

| | | Day 1 | | | Day 7 | | |
|---|---|---|---|---|---|---|---|
| | | Temp | | % | Temp | | % |
| Concentration | Action | ° F. | cP | Torque | ° F. | cP | Torque |
| 3X HMBi | Mix | 70 | 421 | 26.3 | 68 | 427 | 26.7 |
| 4X HMBi | Mix | 70 | 518 | 32.4 | 68 | 568 | 35.5 |
| 3X HMBi | Static | 70 | 402 | 25.1 | 68 | 458 | 28.6 |
| 4X HMBi | Static | 70 | 507 | 31.7 | 68 | 610 | 38.1 |
| 3X HMBi | Mix | 42 | 640 | 40.0 | 34 | 781 | 48.8 |
| 4X HMBi | Mix | 42 | 806 | 50.4 | 34 | 1,011 | 63.2 |
| 3X HMBi | Static | 40 | 666 | 41.6 | 34 | 870 | 54.4 |
| 4X HMBi | Static | 42 | 830 | 51.9 | 34 | 1,083 | 67.7 |

TABLE E1-3b
Time, Concentration of HMBi, and Action Effects on
Viscosity and Percent Torque.

| | | Day 14 | | | Day 21 | | |
|---|---|---|---|---|---|---|---|
| | | Temp | | % | Temp | | % |
| Concentration | Action | ° F. | cP | Torque | ° F. | cP | Torque |
| 3X HMBi | Mix | 72 | 400 | 25.0 | 64 | 400 | 25.0 |
| 4X HMBi | Mix | 72 | 682 | 42.6 | 65 | 528 | 33.3 |
| 3X HMBi | Static | 72 | 482 | 30.1 | 65 | 587 | 36.7 |
| 4X HMBi | Static | 72 | 630 | 39.4 | 66 | 720 | 45.0 |
| 3X HMBi | Mix | 32 | 830 | 51.9 | 30 | 766 | 47.9 |
| 4X HMBi | Mix | 32 | 1,018 | 63.6 | 28 | 1,066 | 66.6 |
| 3X HMBi | Static | 32 | 934 | 58.4 | 28 | 994 | 62.1 |
| 4X HMBi | Static | 32 | 1,178 | 73.6 | 28 | 1,282 | 80.1 |

TABLE E1-3c
Time, Concentration of HMBi, and Action Effects on
Viscosity and Percent Torque.

| | | Day 29 | | | Day 35 | | |
|---|---|---|---|---|---|---|---|
| | | Temp | | % | Temp | | % |
| Concentration | Action | ° F. | cP | Torque | ° F. | cP | Torque |
| 3X HMBi | Mix | 67 | 379 | 23.7 | 67 | 374 | 23.4 |
| 4X HMBi | Mix | 67 | 491 | 30.7 | 66 | 493 | 30.8 |
| 3X HMBi | Static | 67 | 594 | 37.1 | 66 | 650 | 40.6 |
| 4X HMBi | Static | 67 | 796 | 49.4 | 66 | 830 | 51.9 |
| 3X HMBi | Mix | 29 | 771 | 48.2 | 30 | 794 | 49.6 |
| 4X HMBi | Mix | 30 | 1,066 | 66.6 | 29 | 1,082 | 67.6 |
| 3X HMBi | Static | 32 | 1,003 | 62.7 | 30 | 1,005 | 62.8 |
| 4X HMBi | Static | 28 | 1,302 | 81.4 | 30 | 1,309 | 81.8 |

TABLE E1-3d
Time, Concentration of HMBi, and Action Effects on
Viscosity and Percent Torque.

| | | Day 42 | | | Day 50 | | |
|---|---|---|---|---|---|---|---|
| | | Temp | | % | Temp | | % |
| Concentration | Action | ° F. | cP | Torque | ° F. | cP | Torque |
| 3X HMBi | Mix | 68 | 368 | 23.0 | 68 | 371 | 23.2 |
| 4X HMBi | Mix | 68 | 472 | 29.5 | 68 | 488 | 30.7 |
| 3X HMBi | Static | 68 | 682 | 42.6 | 68 | 859 | 53.7 |
| 4X HMBi | Static | 68 | 842 | 52.6 | 68 | 957 | 59.8 |
| 3X HMBi | Mix | 30 | 725 | 45.3 | 32 | 744 | 46.5 |
| 4X HMBi | Mix | 30 | 952 | 59.5 | 32 | 1,037 | 64.8 |
| 3X HMBi | Static | 30 | 992 | 62.0 | 32 | 986 | 61.6 |
| 4X HMBi | Static | 30 | 1,290 | 80.6 | 32 | 1,309 | 81.8 |

TABLE E1-3E
Time, Concentration of HMBi, and Action
Effects on Viscosity and Percent Torque.

| | | Day 56 | | |
|---|---|---|---|---|
| | | Temp | | % |
| Concentration | Action | ° F. | cP | Torque |
| 3X HMBi | Mix | 70 | 354 | 22.1 |
| 4X HMBi | Mix | 69 | 474 | 29.6 |
| 3X HMBi | Static | 69 | 883 | 55.2 |
| 4X HMBi | Static | 70 | 922 | 57.6 |
| 3X HMBi | Mix | 32 | 749 | 46.8 |
| 4X HMBi | Mix | 33 | 1,006 | 62.9 |
| 3X HMBi | Static | 33 | 990 | 61.9 |
| 4X HMBi | Static | 34 | 1,250 | 78.1 |

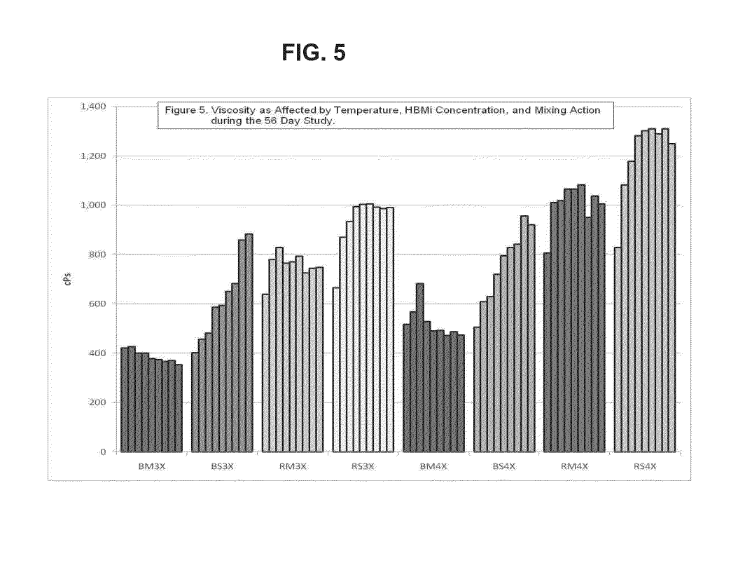

The 3× HMBi was thinner at all temperatures compared to the 4× HMBi samples. Lower torque values were generally seen with the lower concentration HMBi at both room and refrigerator temperatures. For both the 3× and 4× HMBi, stirring approximately three times per week, decreased viscosity and percent torque making the samples more flow able. It was also noted that both concentrations would have been pumpable at the freezing mark. Pumping difficulty arises when a product has a viscosity over 5,000 cP using a sO5 spindle which has a diameter slightly shorter than a nickel or 2 cm. FIG. 5 shows the decrease in viscosity over the study period when the samples were mixed compared to the mixed (M) or static (S) samples by HMBi concentration (3× or 4×) and temperatures (B=bench or room temperature and R=refrigerator temperature).

Each bar represents a data collection day: 1, 7, 14, 21, 29, 35, 42, 50, and 56 days. These data indicated that the HMBi did not cause thickening of the simple mill liquid product to the point of causing concern with flow or pumping in a feed mill setting. Mixing or re-circulation every other day will also maintain positional and product stability plus decrease product viscosity. Additionally, lower temperatures were tried for the 3× and 4× plus the HMBi straight or 100% additive after the 56 day study. At less than 0° F. or −17.8° C. for a period of 24 hours, both the 3× and 4× were still very flow able. However, the 100% HMBi was frozen solid. The liquid 100% HMBi freezes or solidifies at temperatures below 54° F. or 12° C. when the HMBi liquid is acted upon by an outside force such as mixing or pumping. This “Superfusion” property of the liquid 100% HMBi requires expensive heated storage equipment and plumbing to allow for use in a feed mill environment. After 72 hours in a deep freeze, the 3× and 4× also froze solid. However, within hours of removing from the deep freezer, the 3× sample was very flow able at 41° F. or 5° C. and the 4× sample was at 32° F. or 0° C. with the consistency of soft-serve ice cream. The HMBi did not return to a liquid state until about 12 hours later or until it reached a temperature of approximately 56° F. or 13° C. Successive freeze/thawing of the liquid 100% HMBi indicated that the superfusion occurred quicker and it took longer to thaw out to a usable consistency.

Therefore, it became apparent that the simple liquid mill product was disrupting the superfusion property of the straight 100% liquid HMBi. This allows for the elimination of the expensive heating equipment and plumbing system required to handle the straight HMBi. To verify this Example 2 study was undertaken.

### Example 2

### Example E2

A range of inclusion rates of the HMBi was used in the second example to further investigate the reduction or retardation of the superfusion property of the methionine analog (HMBi) added to a simple feed mill blend molasses liquid product. Table E2-1 shows the inclusion ranges on the ingredients used that are similar to the ones used in Example E1.

TABLE E2-1
Liquid Mill Product Formulation and
Methionine Analogue Additions

| Ingredient | Inclusion Range % |
|---|---|
| Molasses Product | 0-45 |
| Cheese Co-Products | 0-23 |
| Wet Corn-Milling Co-Product | 0-17 |
| Ethanol Co-Product | 0-17 |
| Methionine Analogue, HMBi | 1.75 to 100 |

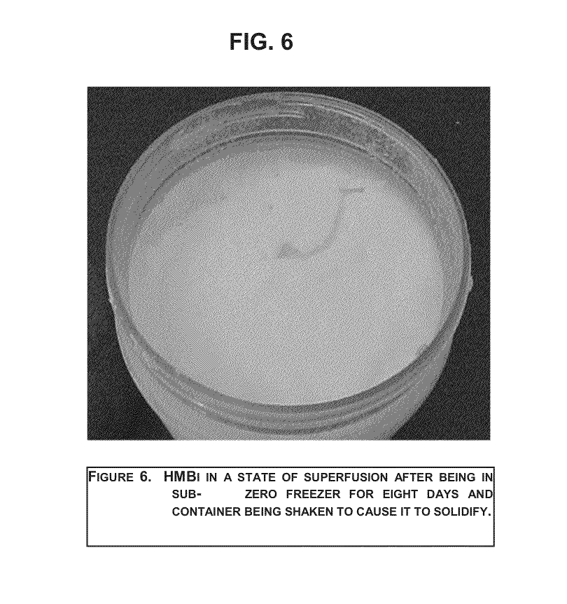

Eight formulations were evaluated in regards to product positional stability, product pH, temperature, viscosity as affected by temperature and time. Viscosity measurements were being used to determine flow ability thus whether or not the product can be pumped without external heating. If HMBi is stored in conditions where its temperature drops below 54° F. (12.2° C.), it will solidify or undergo superfusion which will not allow it to be pumped. As you can see in FIG. 6, the HMBi is solid and will remain solid for several hours while thawing. During the thawing process, the HMBi will liquefy with a solid ball surrounded by liquid. This solid ball could potentially plug the inside fixture of a tank not allowing for flow to the pump. During the Example E2 study, it took about eight days of the 100% HMBi in sub-zero temperatures before it underwent superfusion. The container in FIG. 6 had to be shaken quite vigorously to get the HMBi to solidify. It was not moved until the late afternoon of day 7. By the morning of day 8, it was solid. This event showed the unpredictable and unexpected nature of the superfusion characteristic of HMBi.

Eight formulations are given in Table E2-2. All ingredient inclusions rates were adjusted to accommodate HMBi concentrations. The HMBi concentrations ranged from 0% for the control simple feed mill liquid to 100%. The 100% HMBi was used to verify its superfusion property and to record all observable and recordable observations obtained with this sample. The industry inclusion rate average of HMBi when incorporated into a liquid feed ingredient formulation is generally but in the range of 50 lbs (2.5%) per ton or less.

TABLE E2-2
Liquid Feed Mill Product Test Formulations

| Formulation | Percent Inclusion | | Actual/ton, lbs |
|---|---|---|---|
| F1 | 0% | HMBi | 0 |
| F2 | 1.75% | HMBi | 35 |
| F3 | 4.40% | HMBi | 88 |
| F4 | 8.80% | HMBi | 176 |
| F5 | 13.2% | HMBi | 264 |
| F6 | 17.6% | HMBi | 352 |
| F7 | 25.00% | HMBi | 500 |
| F8 | 100.00% | HMBi | 2,000 |

The containers used were clear PET plastic one quart capacity with wide mouth openings. All samples were placed in a sub-zero freezer until the samples were solid. As stated above, it took eight days for the 100% HMBi to undergo the superfusion process, thus all eight samples were frozen solid at about −9° F. (−22.8° C.) which is exactly what was needed for Example E2.

Once all samples were frozen, they were moved to a force-draft fume hood to allow warm air to be drawn over the jars to hasten thawing. When the samples were soft enough to get a metal tipped thermometer into the samples, temperatures were taken and recorded. Viscosity was measured once the samples thawed enough; temperatures were also taken at that time. The process of freezing and thawing was repeated once. These samples were not stirred or shaken except for the 100% HMBi sample to get it to undergo superfusion.

All formulated samples were mixed by the procedures and using the equipment described in Example E1. The same temperature probe, viscometer, and pH meter were also used.

### Observations and Results:

Observations and data were collected when the samples had thawed enough to allow for viscosity and temperature measurements. The freezing and thawing was repeated once to confirm results. Laboratory room temperature was between 68 to 70° F. Any product having a viscosity over 5,000 cPs using a spindle sO5 will not flow nor be pumpable without added heat.

Table E2-3 has the viscosity data, spindle size, temperature, dates, and times data was collected for each treatment. Viscosity and temperature were measured as describe in Example E1. The initial viscosities at an average of 78.5° F. were similar to those seen in Example E1. As the concentration of HMBi increased in the liquid, its viscosity also increased. At 0.0° F., all samples were too thick to pump as seen in Table E2-3 on January 1. However, the 100% HMBi sample was not solid with a viscosity reading of 174 cP at 1.3° F. Viscosities continued to increase as the temperature dipped to −2.9° F. with the 25% formulation turning solid at −2.5° F. It took until the afternoon of January 11 for the 100% HMBi formulation to solidify. The thawing process was started on January 14 to collect data on the other samples. At an average temperature of 13.5° F. (range of 9.1 to 20.1° F.), all formulations were flow able. The spindle used on January 14 at 3:00 pm was sO3. At the same data collection time, the 100% HMBi was solid thus no temperature or viscosity reading was obtained. An hour later (3:50 to 4:15 pm), all viscosities had dropped by a minimum 65% with an average increase in temperature of approximately 12° F. to 25° F. The 100% HMBi sample was still solid. Over the next hour, the average temperature rose to 37° F. with a further decrease in viscosity. The 100% HMBi was now at a semi-solid state registering 47.7° F., but would still not be pumpable. This was also the case two hours later, 7:48 to 8:30 pm on January 14 at which time the temperature was 62.3° F. At 8:30 pm, all samples were returned to the deep freeze to repeat the process.

The next morning at about 11:00 am the 100% HMBi was solid. Apparently, once HMBi undergoes superfusion, it solidifies much quicker. The 1.75, 8.8, 17.6 and 25% HMBi samples were all still pump able at approximately 1.0° F. The other formulations were not pumpable. By noon on January 15, all samples were flowable and pumpable with an average temperature of 31° F. But the 100% HMBi was at a semi-solid state. From 4:19 pm to 4:51 pm on January 15, all samples viscosities were slightly higher than the initial readings the day before but the temperature was lower, averaging 65° F. The 100% HMBi temperature was 45° F. at a semi-solid state.

Phase IV MetaSmart Methionine Mill Liquid Product Testing Freeze/Thaw Study

| | TREATMENTS | | | | | | | |
|---|---|---|---|---|---|---|---|---|
| DATE | 0% | 1.75% | 4.40% | 8.80% | 13.20% | 17.60% | 25% | 100% |
| Dec. 31, 2009 | | | | | | | | |
| lbs/ton | 0 | 35 | 88 | 176 | 264 | 352 | 500 | 2000 |
| cPs | 113 | 141 | 198 | 295 | 515 | 646 | 851 | 37 |
| Spindle Size | sO1 | sO1 | sO1 | sO1 | sO2 | sO2 | sO2 | sO1 |
| Temp, ° F. | 78.4 | 77.7 | 78.5 | 78.8 | 79.0 | 78.9 | 79.6 | 77.3 |
| Jan. 1, 2010 | | | | | | | | |
| cPs | 5,200 | 6,200 | 7,360 | 11,320 | 7,000 | 9,280 | 15,600 | 173.6 |
| Spindle Size | sO6 | sO6 | sO6 | sO6 | sO6 | sO6 | sO6 | sO1 |
| Temp, ° F. | 0.4 | 0.9 | 0.1 | −0.3 | −0.5 | 0.0 | −1.0 | 1.3 |
| Jan. 2, 2010 | | | | | | | | |
| cPs | 15,680 | 20,480 | 24,360 | 28,440 | 22,000 | 30,200 | | 157.6 |
| Spindle Size | sO6 | sO6 | sO6 | sO6 | sO6 | sO6 | SOLID | sO1 |
| Temp, ° F. | −1.8 | −3.7 | −3.9 | −3.9 | −3.1 | −3.7 | −2.5 | −0.2 |
| Jan. 14, 2010 | 2:59:00-3:34 PM | | | | | | | |
| cPs | 824 | 900 | 852 | 3,548 | 3,428 | 5,530 | 13,430 | |
| Spindle Size | sO3 | sO3 | sO3 | sO3 | sO3 | sO3 | sO3 | SOLID |
| Temp, ° F. | 9.1 | 9.3 | 9.1 | 14.5 | 16.0 | 16.3 | 20.1 | |
| Jan. 14, 2010 | 3:50:00-4:15 PM | | | | | | | |
| cPs | 258 | 387 | 426 | 933 | 1,283 | 2,379 | 4,347 | |
| Spindle Size | sO2 | sO2 | sO2 | sO2 | sO2 | sO2 | sO3 | SOLID |
| Temp, ° F. | 26.1 | 19.4 | 25.7 | 22.3 | 24.6 | 26.1 | 30.6 | |
| Jan. 14, 2010 | 4:40-5:10 PM | | | | | | | |
| cPs | 179 | 296 | 270 | 498 | 658 | 882 | 1,484 | |
| Spindle Size | sO1 | sO1 | sO1 | sO1 | sO2 | sO2 | sO3 | SEMI-SOLID |
| Temp, ° F. | 41.2 | 35.0 | 39.4 | 34.0 | 35.7 | 36.6 | 37.5 | 47.7 |
| Jan. 15, 2010 | 10:55-11:20 AM | | | | | | | |
| cPs | 6,120 | 3,330 | 6,350 | 3,630 | 5,940 | 3,760 | 5,100 | |
| Spindle Size | sO6 | sO5 | sO5 | sO5 | sO5 | sO5 | sO5 | SOLID |
| Temp, ° F. | 0.5 | 2.1 | 1.0 | 0.7 | 1.8 | 0.4 | −0.1 | |
| Jan. 15, 2010 | 12:05-12:27 NOON | | | | | | | |
| cPs | 289 | 422 | 325 | 392 | 531 | 1,437 | 973 | |
| Spindle Size | sO1 | sO2 | sO2 | sO2 | sO2 | sO2 | sO2 | SOLID |
| Temp, ° F. | 25.3 | 18.0 | 31.6 | 32.4 | 34.7 | 32.4 | 41.0 | |
| Jan. 15, 2010 | 4:19-4:51 PM | | | | | | | |
| cPs | 140 | 178 | 183 | 262 | 366 | 513 | 662 | |
| Spindle Size | sO1 | sO1 | sO1 | sO1 | sO1 | sO1 | sO1 | SEMI-SOLID |
| Temp, ° F. | 63.3 | 63.8 | 65.2 | 64.4 | 65.4 | 64.4 | 66.0 | 45.0 |

Made on Dec. 29, 2009

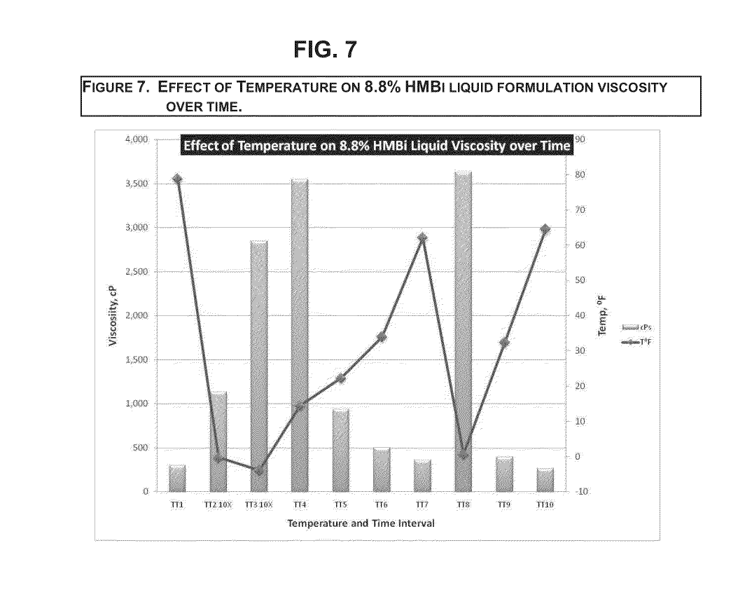

FIG. 7 shows time and temperature affects on the viscosity of the 8.8% HMBi formulation. It can be seen from this graph that there is a temperature minimum above which this formulation would remain flowable and pumpable. That minimum temperature would be approximately 5° F. TT2 and TT3 viscosities are 10 times the values shown on the graph, 11,320 and 28,440 cP with spindle sO6, respectively. The factor-10 was used to keep the bars for TT2 and TT3 on the graph page. At TT8, this formulation was still flowing indicating the liquid blend was not allowing the HMBi to undergo superfusion.

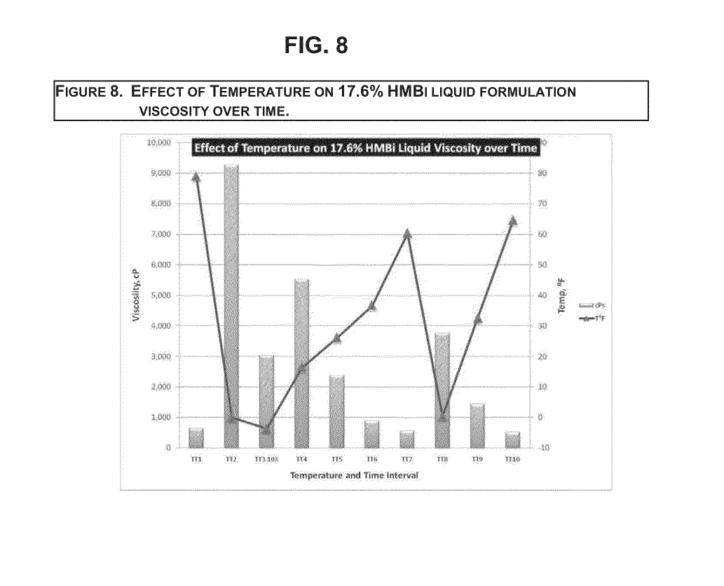

This can also be seen in FIG. 8 with the 17.6% HMBi formulation. But it appears the minimum temperature needs to be about 20° F. with twice as much HMBi in the formulation. This is well below the 12° C. (54° F.) minimum recommended by the manufacturer of the HMBi for storage and handling. It also appears, that with successive freeze-thaw events, as the HMBi concentration increases in the mill liquid, the product will have lower viscosities thus better handling in regards to flowability and pumping.

Thus with HMBi concentrations ranging from 4.4% to 25% incorporated into a simple feed mill blend liquid supplement as previously described and keeping that liquid at approximately −1° C. (30° F.), the liquid would remain flowable and allow for pumping without the need for expensive heated storage containers and equipment.

It is specifically intended that the present invention not be limited to the embodiments and illustrations contained herein, but include modified forms of those embodiments including portions of the embodiments and combinations of elements of different embodiments as come within the scope of the following claims.

We claim:

1. A composition comprising an intimate admixture of high concentrations of a liquid methionine product and a liquid mill/feed product where the crystallization temperature of the admixture is suppressed at least 10° F. relative to the crystallization temperature of liquid methionine product before admixing.

2. A composition according to claim 1 wherein the admixture crystallization temperature is suppressed at least 18° F.

3. A composition according to claim 1 wherein the admixture crystallization temperature is suppressed at least 24° F.

4. A method of suppressing the crystallization temperature of an admixture product comprising the steps of:
providing a high concentration of a liquid methionine product;
providing a liquid feed/mill product;
admixing the high concentration of liquid methionine product with the liquid feed/mill product to create an intimate admixture, the crystallization temperature of the admixture product is suppressed (Δt_{c}) at least 10° F. relative to the liquid methionine product crystallization.

5. A method according to claim 4 wherein the crystallization temperature of the liquid methionine is suppressed at least 18° F.

6. A method according to claim 4 wherein the crystallization temperature of the liquid methionine is suppressed at least 24° F.

7. A composition comprising an admixture of a liquid methionine product and a liquid feed/mill product in which the crystallization temperature of the liquid methionine product is suppressed at least 24° F. and where the weight ratio of the liquid methionine product to the liquid feed mill product falls in the range of about 4% to 25%.

8. A method of administering a liquid amino acid having a crystallization temperature of 54° F. (12° C.) or below to a bovine in nutritional need of the amino acid comprising the steps of:
providing a high concentration of a liquid methionine product;
providing a liquid feed/mill product;
admixing the high concentration of liquid methionine product with the liquid feed/mill product to create an intimate admixture, the crystallization temperature of the admixture product is suppressed (Δt_{c}) at least 10° F. relative to the liquid methionine product crystallization,
feeding the admixture of the previous sentence to the bovine in an amount sufficient to satisfy the bovine's nutritional need.

9. A method according to claim 8 wherein the bovine is a cow.

10. A method according to claim 8 wherein the bovine is a lactating cow.

11. A method according to claim 8 wherein the amino acid is methionine.

12. A method according to claim 8 wherein the weight ratio of the liquid methionine product to the liquid feed/mill product falls in the range of above 4% to 25%.

13. A method according to claim 8 wherein the amino acid is a methionine analogue.

14. A method according to claim 8 wherein Δt_{c} is 18° F., (10° C.).

15. A method according to claim 8 wherein Δt_{c} is 24° F. (13.3° C.).
